(19) United States
(12) Patent Application Publication
(10) Pub. No.: US 2026/0199895 A1
(43) Pub. Date: Jul. 16, 2026

(54) SMALL-SIZE CHIP FOR NUCLEIC ACID DETECTION AND USE THEREOF

(71) Applicant: EVERLAST HEALTHCARE LIMITED, Hong Kong (CN)

(72) Inventor: Haifeng ZHAO, Beijing (CN)

(21) Appl. No.: 19/132,164

(22) PCT Filed: Nov. 27, 2023
(86) PCT No.: PCT/CN2023/134480

§ 371 Date: Jul. 22, 2025

(30) Foreign Application Priority Data
Nov. 25, 2022 (CN) 202211487626.0

(51) Int. Cl. B01L 3/00; B01L 7/00; C12Q 1/6806; C12Q 1/6844

(52) U.S. Cl. CPC B01L 3/502715; B01L 3/502746; B01L 7/52; C12Q 1/6806; C12Q 1/6844; B01L 2200/0689; B01L 2200/148; B01L 2300/042; B01L 2300/044; B01L 2300/087; B01L 2300/18; B01L 2400/086

(57) ABSTRACT

A chip device for detecting nucleic acid in a sample, includes a substrate, a sample loading chamber, a sample chamber detachably connected to the sample loading chamber, and an amplification reaction chamber arranged in the substrate. The sample chamber is configured to receive a sample to be detected, and be inserted into the sample loading chamber to form fluid communication therewith, wherein the sample to be detected in the sample chamber enters the amplification reaction chamber in the substrate through the sample loading chamber. An apparatus for detecting nucleic acid in a sample, preferably a POCT apparatus, includes the chip device. A method is for detecting nucleic acid in a sample performed by means of the chip device or the apparatus.

### TECHNICAL FIELD

The present invention relates to the field of biotechnology and use of biotechnical device, and specifically to a small-size chip device and apparatus for detecting nucleic acid in samples, and use thereof in biological sample detection.

### TECHNICAL BACKGROUND

Nucleic acid detection is at the center of many fields including clinical trials and identification of pathogenic microorganisms. Various diseases such as cancer, microbial infections and genetic markers can be detected through nucleic acid extraction, amplification and detection analysis.

Methods using PCR and real-time PCR are effective in exponentially amplifying and detecting genes. The application of PCR/real-time PCR devices has rapidly developed on the market for genetic detection of infectious diseases, such as viral, sexually transmitted diseases, influenza or the like. Gene detection is playing a more significant role in the treatment of cancer. However, the automation of applications using PCR and RT-PCR, especially small-size automation, is not easy to realize.

Therefore, there is still a need in the field for high-speed, real-time and small-size nucleic acid amplification and detection device and apparatus that is convenient to operate, and particularly for chip device and apparatus suitable for point-of-care testing (POCT).

### SUMMARY OF THE INVENTION

The present invention proposes a small-size chip device for detecting nucleic acid in a sample, which includes a substrate, a sample loading chamber, a sample chamber detachably connected to the sample loading chamber, and an amplification reaction chamber arranged in the substrate, the sample loading chamber being in communication with the amplification reaction chamber through a liquid flow channel in the substrate,
wherein the sample chamber is configured to receive a sample to be detected, and be inserted into the sample loading chamber to form fluid communication therewith, whereby the sample to be detected in the sample chamber enters the amplification reaction chamber in the substrate through the sample loading chamber; and
wherein the sample chamber includes a needle, so that the sample chamber is in communication with a sample loading pipe of the sample loading chamber through the needle, allowing a solution in the sample chamber to enter the sample loading chamber.

According to one aspect of the present invention, a bottom portion of the sample chamber has an upper sealing rubber plug for sealing the needle, and a top portion of the sample loading chamber has a lower sealing rubber plug for sealing the sample loading pipe,
wherein a lower end of the sample chamber is configured to be inserted into the sample loading chamber after the sample chamber and the sample loading chamber are assembled together, whereby the needle of the sample chamber penetrates the upper sealing rubber plug and the lower sealing rubber plug to be inserted into the sample loading pipe of the sample loading chamber, so that the sample chamber is in fluid communication with the sample loading chamber.

According to one aspect of the present invention, the sample loading chamber includes a spring which is compressed when the sample chamber is inserted into the sample loading chamber,
wherein a sample loading column fixed to the substrate is arranged in the sample loading chamber, and the sample loading pipe is arranged on a lower portion of the sample loading column, a gap being formed between an outer wall of the sample loading chamber and the sample loading column, for receiving the spring and allowing the lower end of the sample chamber to be inserted.

According to one aspect of the present invention, the sample loading chamber and the substrate are restored to a sealed state after a sufficient amount of solution for detection from the sample chamber has been transferred to the amplification reaction chamber in the substrate.

According to one aspect of the present invention, the sample loading chamber and the substrate are restored to the sealed state by withdrawing the needle of the sample chamber from the sample loading pipe of the sample loading chamber,
wherein a tip of the needle of the sample chamber is configured to stay in the lower sealing rubber plug of the sample loading chamber after the needle is withdrawn from the sample loading pipe of the sample loading chamber.

According to one aspect of the present invention, a limiting member, which is a wedge-shaped member with a protruding lower end, is arranged inside the sample loading chamber, wherein the protruding lower end is deformed when subjected to pressure, and
a limiting groove is arranged at a lower bottom end of the sample chamber, the lower end of the limiting member being clamped into the limiting groove of the sample chamber when the sample chamber moves upward to the limiting member.

According to one aspect of the present invention, a cap is provided on a top portion of the sample chamber.

According to one aspect of the present invention, a reagent solution for preserving and extracting the nucleic acid in the sample is added to or pre-provided in the sample chamber, and materials for the nucleic acid amplification reaction are pre-provided in the amplification reaction chamber.

According to one aspect of the present invention, a pressure sharing chamber, which is arranged downstream of the amplification reaction chamber and within the substrate of the chip, is in communication with the amplification reaction chamber through a pressure sharing gas channel.

The present invention also proposes an apparatus for detecting nucleic acid in a sample, comprising said chip device.

According to one aspect of the present invention, the apparatus includes at least one of a chip device receiving system, a signal detection module for detecting nucleic acid amplification products, a temperature control system for a nucleic acid amplification area of the chip device, and an analysis and/or output system for nucleic acid amplification results.

According to one aspect of the present invention, the apparatus is a POCT apparatus.

The present invention further proposes a method for detecting nucleic acid in a sample, which is performed by means of said chip device or said apparatus,
wherein the method includes steps of:
   (1) adding the sample to the sample chamber of the chip device, so that the nucleic acid in the sample is separated to enter the solution;
   (2) inserting the sample chamber into the sample loading chamber to form a fluid communication therebetween, whereby the sample to be detected in the sample chamber enters the amplification reaction chamber in the substrate through the sample loading chamber; and
   (3) carrying out the amplification reaction of the nucleic acid in the solution in the amplification reaction chamber.

According to one aspect of the present invention, the nucleic acid amplification reaction is an isothermal amplification method.

According to one aspect of the present invention, the nucleic acid amplification reaction is loop-mediated isothermal amplification (LAMP) of DNA.

According to one aspect of the present invention, the sample chamber includes a needle, through which the sample chamber is in communication with the sample loading pipe of the sample loading chamber, allowing the solution in the sample chamber to enter the sample loading chamber;

wherein an upper sealing rubber plug for sealing the needle is arranged at the bottom portion of the sample chamber, and a lower sealing rubber plug for sealing the sample loading pipe is arranged at the top portion of the sample loading chamber, so that after the sample chamber and the sample loading chamber are assembled together, the lower end of the sample chamber is inserted into the sample loading chamber, whereby the needle of the sample chamber penetrates the upper sealing rubber plug and the lower sealing rubber plug to be inserted into the sample loading pipe of the sample loading chamber, thereby forming a fluid communication between the sample chamber and the sample loading chamber.

According to one aspect of the present invention, after a sufficient amount of solution for detection from the sample chamber has been transferred to the amplification reaction chamber in the substrate, the sample loading chamber and the substrate are restored to a sealed state by withdrawing the needle of the sample chamber from the sample loading pipe of the sample loading chamber.

According to one aspect of the present invention, a tip of the needle of the sample chamber is controlled to stay in the lower sealing rubber plug of the sample loading chamber after the needle is withdrawn from the sample loading pipe of the sample loading chamber.

According to one aspect of the present invention, a limiting member which is a wedge-shaped member with a protruding lower end, is arranged inside the sample loading chamber, wherein the protruding lower end is deformed when subjected to pressure, and a limiting groove is arranged at a lower bottom end of the sample chamber, the lower end of the limiting member being clamped into the limiting groove of the sample chamber when the sample chamber moves upward to the limiting member.

According to one aspect of the present invention, identifiable markers carried by the amplified nucleic acid are detected through luminescence in the form of fluorescence or one selected from a group consisting of chemiluminescence, bioluminescence, radioluminescence, electroluminescence, electrochemiluminescence, mechanoluminescence, crystalloluminescence, thermoluminescence, sonoluminescence, phosphorescence and photoluminescence, or enzymatic reactions, radioactivity.

### BRIEF DESCRIPTION OF THE DRAWINGS

In order to illustrate the embodiments of the present invention or the technical solutions in the prior arts more clearly, the drawings as required herein will be briefly introduced as follows. Apparently, the drawings as follows show some embodiments of the present invention, based on which other drawings can also be obtained by one skilled in the art without creative labors.

FIG. 1A is a three-dimensional assembly diagram schematically showing an exemplary chip device (or referred to as a chip in this context) for detecting nucleic acid in a sample according to the present invention, which includes a substrate 1, a sample loading chamber 2 vertically arranged on the substrate, a sample chamber 3 detachably connected to the sample loading chamber 2, and an amplification reaction chamber 4 in the substrate of the chip.

FIG. 1B is a three-dimensional perspective view of the chip device for detecting nucleic acid in a sample, wherein the sample loading chamber 2 and the sample chamber 3 are separate from each other.

FIG. 2 is an exploded view showing the chip device for detecting nucleic acid in a sample.

FIGS. 3A, 3B and 3C schematically show the chip device for detecting nucleic acid in a sample in a working state. In FIG. 3A, a sample solution to be detected is added to the sample chamber 3 of the chip device for detecting nucleic acid in a sample. In FIG. 3B, the sample chamber 3 with the sample solution to be detected is inserted into the sample loading chamber 2, wherein the sample chamber 3 is in fluid communication with the sample loading chamber 2 through a needle 34, so that the sample solution to be detected in the sample chamber 3 enters the amplification reaction chamber 4 in the substrate through the sample loading chamber 2. In FIG. 3C, the needle of the sample chamber 3 is withdrawn upwards to stay in a lower sealing rubber plug 25 after a sufficient amount of solution is transferred.

### DETAILED DESCRIPTION OF EMBODIMENTS

In order to illustrate the objectives, technical solutions, and advantages of the embodiments of the present invention more clearly, the technical solutions in the embodiments of the present invention are described below clearly and completely in conjunction with the drawings in the embodiments of the present invention. Obviously, the embodiments described herein are some of, but not all of the embodiments of the present invention. All other embodiments obtained by one skilled in the art without creative labors based on the embodiments in the present invention shall fall within the protection scope of the present invention.

### Embodiment 1

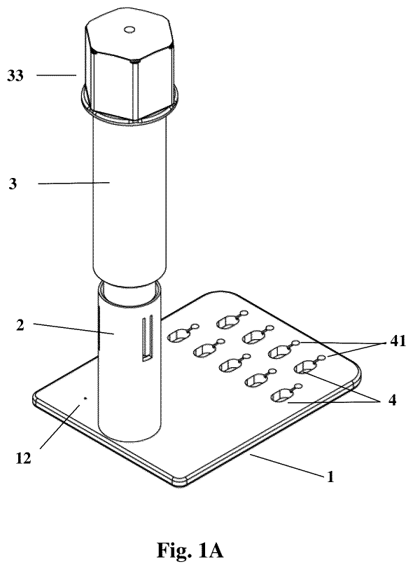

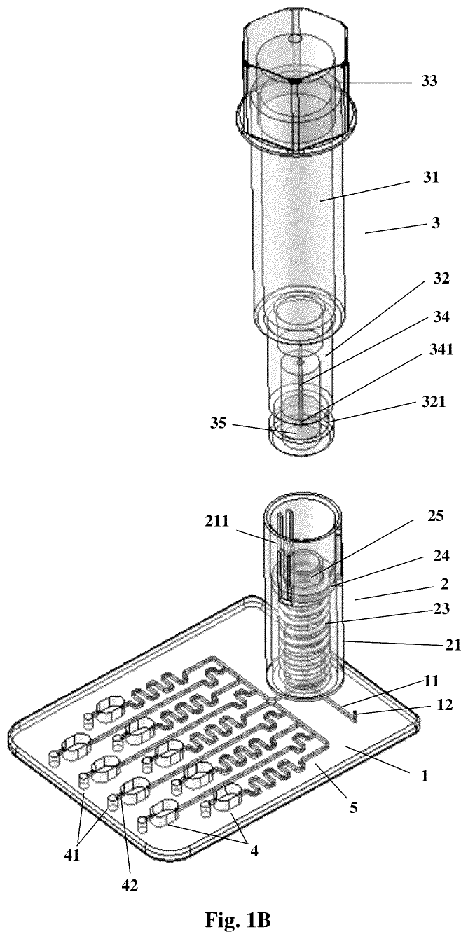

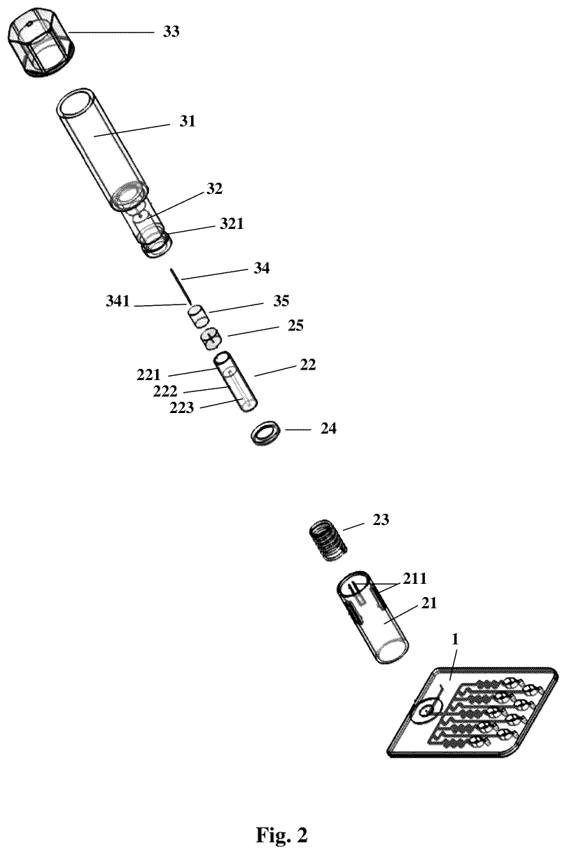

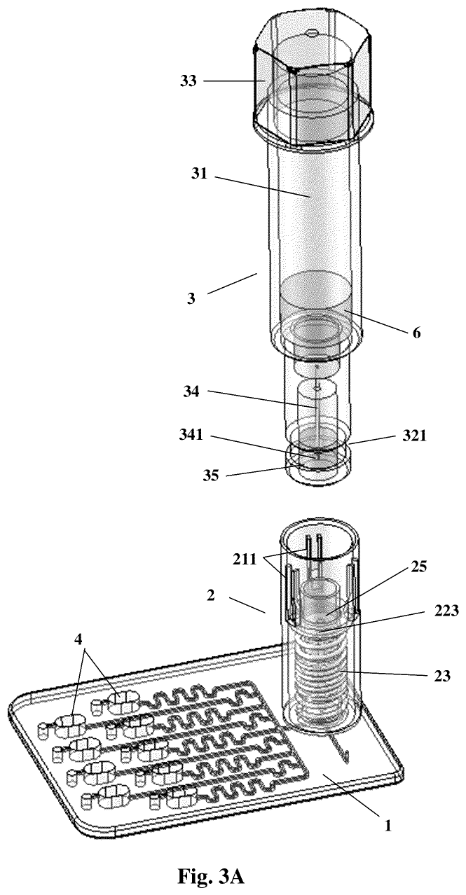

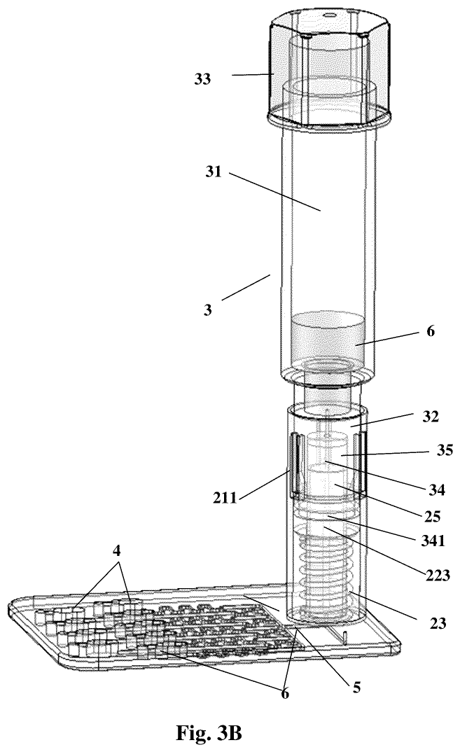

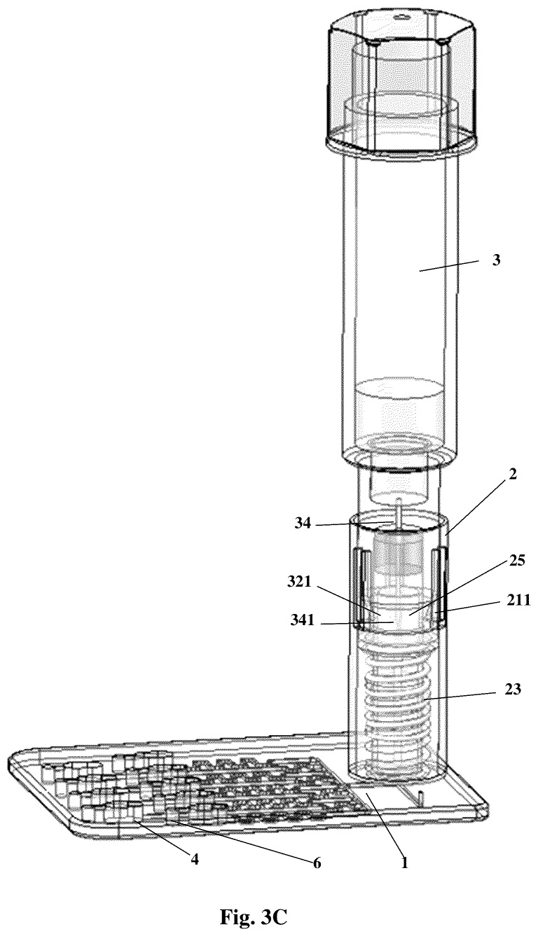

FIG. 1A is a three-dimensional assembly diagram schematically showing an exemplary chip device (or referred to as a chip in this context) for detecting nucleic acid in a sample according to the present invention, which includes a substrate 1, a sample loading chamber 2 vertically arranged on the substrate, a sample chamber 3 detachably connected to the sample loading chamber 2, and an amplification reaction chamber 4 in the substrate of the chip. FIG. 1B is a three-dimensional perspective view of the chip device for detecting nucleic acid in a sample, wherein the sample loading chamber 2 and the sample chamber 3 are separate from each other. FIG. 2 is an exploded view showing the chip device for detecting nucleic acid in a sample. FIGS. 3A, 3B and 3C schematically show the chip device for detecting nucleic acid in a sample in a working state. In FIG. 3A, a sample solution to be detected is added to the sample chamber 3 of the chip device for detecting nucleic acid in a sample. In FIG. 3B, the sample chamber 3 with the sample solution to be detected is inserted into the sample loading chamber 2, wherein the sample chamber 3 is in fluid communication with the sample loading chamber 2 through a needle 34, so that the sample solution to be detected in the sample chamber 3 enters the amplification reaction chamber 4 in the substrate through the sample loading chamber 2. In FIG. 3C, the needle of the sample chamber 3 is withdrawn upwards to stay in a lower sealing rubber plug 25 after a sufficient amount of solution is transferred.

FIG. 1A is a three-dimensional assembly diagram schematically showing an exemplary chip device for detecting nucleic acid in a sample according to the present invention. As shown in FIG. 1A, the chip device for detecting nucleic acid in a sample according to the present invention includes a sample loading chamber 2 vertically arranged on the substrate, a sample chamber 3, and an amplification reaction chamber 4 in the substrate of the chip, wherein the sample loading chamber 2 and the sample chamber 3 are detachable from each other. FIG. 1B is a three-dimensional perspective view of the chip device for detecting nucleic acid in a sample, wherein the sample loading chamber 2 and the sample chamber 3 are separate from each other. FIG. 2 is an exploded view showing the chip device for detecting nucleic acid in a sample.

As shown in FIGS. 1B and 2, the sample loading chamber 2 is a cylindrical chamber fixed to the substrate 1 and encased by an outer wall 21 perpendicular to the substrate. A sample loading column 22 fixed to the substrate 1 is arranged in the sample loading chamber 2. An upper portion 221 of the sample loading column 22 is a hollow chamber for receiving a lower sealing rubber plug 25 therein, and a lower portion 222 thereof includes a sample loading pipe 223 into which a needle 34 of the sample chamber 3 is inserted. A gap is formed between the sample loading column 22 and the outer wall 21 of the sample loading chamber 2, for receiving a spring pressing ring 24 and a spring 23 located below the spring pressing ring 24, i.e. closer to the substrate 1, and also allowing the part of the sample chamber 3 to be assembled with the sample loading chamber 2, such as a lower chamber 32 of the sample chamber 3, to be inserted therein. The sample loading pipe is in communication with a liquid flow channel in the substrate through an opening at a bottom portion of the sample loading chamber. According to one embodiment of the present invention, the sample loading pipe 223 is in communication with a liquid flow channel 5 arranged in the substrate through an opening at a bottom portion of the sample loading column 22.

The amplification reaction chamber 4 is arranged in the substrate of the chip, for containing reactants or a reaction system for the nucleic acid amplification reaction, and a solution containing separated nucleic acids from the sample chamber for the amplification reaction in suitable conditions. According to other aspects of the present invention, the amplification reaction chamber of the chip device includes a plurality of, such as 2 to 48 or more, nucleic acid amplification units.

As shown in FIGS. 1B and 2, the sample chamber 3 is a cylindrical container for receiving a sample to be detected, and can be inserted into the sample loading chamber 2 for fluid communication therebetween. Therefore, the sample to be detected in the sample chamber 3 can enter the amplification reaction chamber 4 in the substrate through the sample loading chamber 2. A top portion of the sample chamber 3 includes a cap 33 that can be opened or closed. According to one embodiment of the present invention, the cap 33 may have internal threads for engaging with external threads formed on an upper portion of the sample chamber 3, thus providing a tight seal for the sample chamber, in order to avoid gas or liquid leakage.

In this embodiment, the sample chamber 3 is divided into an upper chamber 31 and the lower chamber 32. The upper chamber 31 of the sample chamber 3 is a hollow chamber for containing the sample to be detected (e.g., a liquid sample or a solution for processing a solid sample), with the cap 33 that can be opened or closed at a top portion thereof. An outer diameter of the upper chamber 31 is the same as that of the outer wall 21 of the sample loading chamber 2. An outer diameter of the lower chamber 32 of the sample chamber 3 is smaller than that of the upper chamber 31, so that the lower chamber 32 can be inserted into a cavity encased by the outer wall 21 of the sample loading chamber 2.

The needle 34 is arranged in the lower chamber 32 of the sample chamber 3. An upper sealing rubber plug 35 is arranged in a distal chamber of the lower chamber 32. One open end of the needle 34 is in the upper chamber 31, or at a junction between the upper chamber 31 and the lower chamber 32. When the solution is added to the upper chamber, it can enter the needle 34. The other end of the needle 34 is a sharp tip 341, which is located in the upper sealing rubber plug 35 in an initial state. Thus, the upper sealing rubber plug 35 can seal the needle 34. After the sample to be detected is added into the sample chamber 3, the opening at the top portion thereof is sealed by the cap 33, and the needle 34 is sealed by the upper sealing rubber plug 35, so that the sample chamber 3 forms an independent sealed structure.

As mentioned above, the sample loading column 22 fixed to the substrate 1 is arranged in the sample loading chamber 2. The upper portion 221 of the sample loading column 22 is a hollow chamber for receiving the lower sealing rubber plug 25, and the lower portion 222 thereof includes the sample loading pipe 223 into which the needle 34 of the sample chamber 3 is inserted. In the initial state, i.e. when the needle 34 of the sample chamber is not inserted, the lower sealing rubber plug 25 seals the sample loading pipe 223, whereby the sample loading chamber 2 and the substrate 1 (including the amplification reaction chamber 4 within the substrate of the chip) form an independent sealed structure.

Therefore, when the sample chamber 3 and the sample loading chamber 2 in the chip device for detecting nucleic acid in a sample according to the present invention are separate from each other, the sample chamber 3 forms an independent sealed structure, and the sample loading chamber 2 and the substrate 1 (including the amplification reaction chamber 4 within the substrate of the chip) also form an independent sealed structure.

In operation, the sample chamber 3 can be in communication with the sample loading chamber 2 through the needle 34, allowing the solution in the sample chamber to enter the sample loading chamber 2 and then enter the amplification reaction chamber 4 in the substrate through the liquid flow channel 5 in the substrate.

In an exemplary operation, the sample chamber 3 is inserted into the sample loading chamber 2, so that a bottom portion of the lower chamber 32 of the sample chamber 3 is in contact with the upper portion 221 of the sample loading column 22 of the sample loading chamber 2, and the upper sealing rubber plug 35 of the sample chamber 3 is in contact with the lower sealing rubber plug 25 of the sample loading chamber 2. When the sample chamber 3 continues to move downward (i.e. toward the substrate), the sharp tip 341 of the needle can penetrate the upper sealing rubber plug 35, enter and penetrate the lower sealing rubber plug 25, and then be inserted into the sample loading pipe 223 of the sample loading chamber 2, so that the sample chamber 3 is in fluid communication with the sample loading chamber 2. The sample loading chamber 2 and the substrate 1 (including the amplification reaction chamber 4 within the substrate of the chip) initially form an independent sealed structure, wherein the chambers and flow channels therein remain in a vacuum state. When the needle of the sample chamber 3 is inserted into the sample loading pipe 223 of the sample loading chamber 2, the sample chamber 3 is in fluid communication with the sample loading chamber 2. Due to the air pressure, the solution in the sample chamber enters the sample loading chamber 2 and then enters the amplification reaction chamber 4 in the substrate through the liquid flow channel 5 in the substrate.

After a sufficient amount of solution for detection has been transferred from the sample chamber 3 to the amplification reaction chamber 4 in the substrate, it is desirable to restore the sample loading chamber 2 and the substrate 1 (including the amplification reaction chamber 4 in the substrate of the chip) to a sealed state. According to one aspect of the present invention, the sample loading chamber 2 and the substrate 1 can be restored to the sealed state by withdrawing the needle of the sample chamber 3 from the sample loading pipe 223 of the sample loading chamber 2. When the needle of the sample chamber 3 leaves the sample loading pipe 223 of the sample loading chamber 2, the lower sealing rubber plug 25 can seal the sample loading pipe 223.

According to one aspect of the present invention, the needle 34 of the sample chamber 3 may be withdrawn from the sample loading pipe 223 of the sample loading chamber 2 manually.

According to another aspect of the present invention, the needle 34 of the sample chamber 3 can be withdrawn from the sample loading pipe 223 of the sample loading chamber 2 through a mechanical mechanism. According to one embodiment of the present invention, a gap is formed between the outer wall 21 of the sample loading chamber 2 and the sample loading column 22, for receiving the spring 23 and the spring pressing ring 24. When the sample chamber 3 is inserted into the sample loading chamber 2, the lower chamber 32 of the sample chamber 3 enters the gap between the sample loading column 22 and the outer wall 21 of the sample loading chamber 2. After the lower chamber 32 is in contact with the spring pressing ring 24, the spring 23 is compressed when the lower chamber 32 of the sample chamber 3 continues to move toward the substrate. When the lower chamber 32 of the sample chamber 3 moves toward the substrate to an appropriate position, the sample chamber stops to move downward, for example, by removing a downward pressure on the sample chamber. (Said appropriate position may be a specified position, i.e. a pre-measured or preset position. When a bottom portion of the lower chamber 32 of the sample chamber 3 reaches this position, the amount of the solution transferred from the sample chamber 3 to the amplification reaction chamber 4 in the substrate is sufficient for detection.) At this time, the spring 23 is extracted, thereby pushing the lower chamber 32 of the sample chamber 3 (including the needle 34) upwards.

According to yet another aspect of the present invention, it is desirable to control the position where the needle of the sample chamber 3 stays after it is withdrawn from the sample loading pipe 223 of the sample loading chamber 2. According to one aspect of the present invention, the position where the needle of the sample chamber 3 stays is controlled through a limiting mechanism. In the exemplary embodiment as shown in the drawings, a limiting member 211 is arranged inside the outer wall 21 of the sample loading chamber 2. The limiting member is a wedge-shaped member with a protruding lower end, which will be deformed, in particular, contracted when subjected to pressure. Meanwhile, a limiting groove 321 is arranged at the bottom portion of the lower chamber 32 of the sample chamber 3. When the sample chamber 3 is inserted into the sample loading chamber 2 under a thrust and moves downward (toward the substrate), the limiting member 211 is deformed when the sample chamber 3 passes over the limiting member 211, but the downward movement of the sample chamber 3 is not limited. When the sample chamber 3 moves upward, for example, when it is pushed upward by the spring 23, to the limiting member 211 of the sample loading chamber, the lower end of the limiting member 211 is clamped into the limiting groove 321 of the sample chamber, thus restricting the position of the sample chamber 3.

According to yet another aspect of the present invention, it is desirable to control the tip 341 of the needle in the sample chamber 3 to stay in the lower sealing rubber plug 25 after the needle in the sample chamber 3 is withdrawn upward from the sample loading pipe 223 of the sample loading chamber 2. Thus, the lower sealing rubber plug 25 seals the sample loading pipe 223, and the sample loading chamber 2 and the substrate 1 (including the amplification reaction chamber 4 within the substrate of the chip) form a sealed structure. In the meantime, the lower sealing rubber plug 25 seals the needle 34, and the sample chamber 3 remains to be sealed. According to yet another aspect of the present invention, said limiting mechanism in the chip enables the tip 341 of the needle in the sample chamber 3 to stay in the lower sealing rubber plug 25 after the needle in the sample chamber 3 is withdrawn upward from the sample loading pipe 223 of the sample loading chamber 2.

According to one aspect of the present invention, a reagent solution for preserving (so as to avoid undesirable decomposition of the nucleic acid to be detected) and extracting nucleic acid in the sample can be added or pre-provided in the sample chamber 3.

When the sample needs to be added, the cap 33 of the sample chamber 3 can be opened. After the sample is added to the sample chamber, the sample chamber is sealed by the cap, so as to avoid the leakage of biological samples and the resulting contamination.

When a liquid sample is added to or stored in the sample chamber 3 in the chip for detecting nucleic acid according to the present invention, the sample chamber 3 is aligned with and inserted into the sample loading chamber 2 and moves toward the substrate by exerting a downward force thereon (for example, by exerting a downward pressure on the cap 33 manually or mechanically). After the lower chamber 32 of the sample chamber 3 is in contact with the upper portion 221 of the sample loading column 22 of the sample loading chamber 2, the sharp tip 341 of the needle of the sample chamber 3 penetrates the upper sealing rubber plug 35, enters and penetrates the lower sealing rubber plug 25, and is then inserted into the sample loading pipe 223 of the sample loading chamber 2, so that the sample chamber 3 is in fluid communication with the sample loading chamber 2. The sample loading chamber 2 and the substrate 1 (including the amplification reaction chamber 4 within the substrate of the chip) initially form an independent sealed structure, wherein the chambers and flow channels therein remain in a vacuum state. When the needle of the sample chamber 3 is inserted into the sample loading pipe 223 of the sample loading chamber 2, the sample chamber 3 is in fluid communication with the sample loading chamber 2. Due to the air pressure, the solution in the sample chamber enters the sample loading chamber 2, and then enters the amplification reaction chamber 4 in the substrate through the liquid flow channel 5 in the substrate.

According to one aspect of the present invention, a distance at which the sample chamber 3 moves in the sample loading chamber 2 to transfer a target volume (a volume sufficient to complete nucleic acid detection) of solution from the sample chamber to the amplification reaction chamber can be determined according to calculations or experiments.

According to one aspect of the present invention, a filtration chamber for filtering out unwanted substances (such as cells, cell debris, or large protein molecules, etc.) in the solution containing the nucleic acid sample can be arranged in the liquid flow channel 5.

Nucleic acid amplification reactions can be carried out in the amplification reaction chamber 4 of the chip device for detecting nucleic acid in a sample according to the present invention. An isothermal amplification method such as LAMP may be adopted. The chip device further includes a temperature control unit for the amplification reaction chamber 4, such as a temperature regulator, in order to keep the amplification reaction chamber at a constant temperature.

According to one aspect of the present invention, a pressure sharing chamber 41 is further provided downstream of the amplification reaction chamber 4 (downstream of a direction in which the gas flows). The pressure sharing chamber, which is provided in the substrate of the chip, is in communication with the amplification reaction chamber 4 through a pressure sharing gas channel 42. The pressure sharing chamber can not only bear the pressure when the air in the amplification reaction chamber is compressed to avoid an adverse impact on the chip due to excessive pressure, but also prevent an area of the amplification reaction chamber from becoming too large, which may affect the efficiency of nucleic acid amplification and signal observation. An opening where the pressure sharing gas channel 42 is connected to the pressure sharing chamber or the amplification reaction chamber is provided at a top portion of the pressure sharing chamber or the amplification reaction chamber.

According to one aspect of the present invention, the sample loading chamber 2 and the substrate 1 (including the amplification reaction chamber 4 within the substrate of the chip) in the chip device for detecting nucleic acid in a sample according to the present invention initially form an independent sealed structure, wherein the chambers and flow channels therein remain in a vacuum state. According to yet another aspect of the present invention, a processing flow channel 11 for evacuating during the preparation of the chip is provided in the substrate of the chip device for detecting nucleic acid in a sample. An opening at one end of the processing flow channel 11 is in communication with the sample loading chamber 2 (such as the sample loading column 22) or the liquid flow channel 5 in the substrate, and an opening 12 at the other end thereof is on the surface of the substrate, which is sealed after the evacuation is completed.

FIGS. 3A, 3B and 3C schematically show operating procedures of the chip device for detecting nucleic acid in a sample.

In an exemplary scenario, the operating procedures of the chip device for detecting nucleic acid in a sample are illustrated as follows.

FIG. 3A shows that the sample chamber 3 already contains the sample solution 6 to be detected, and the sample loading chamber 2 and the sample chamber 3 are still separate from each other. As shown in FIG. 3A, the sample chamber 3 of the chip device for detecting nucleic acid in a sample contains the sample solution 6 to be detected. The sample to be detected may be a liquid sample or a solution for processing a solid sample. An exemplary approach to add and process the sample to be detected includes the following steps. A processing solution such as a sample preservation solution is pre-provided in the upper chamber 31 of the sample chamber. The cap 33 of the sample chamber 3 is opened to add the sample to be detected (such as a throat swab) in the sample chamber 3. A nucleic acid separation reaction is conducted in the sample preservation solution, so that the nucleic acids are released into the solution. Then the cap 33 is put on to seal the opening at the top portion of the sample chamber. The sharp tip 341 of the needle 34 is located in the upper sealing rubber plug 35, so that the sample chamber 3 forms an independent sealed structure.

In the meantime, the lower sealing rubber plug 25 of the sample loading chamber 2 seals the sample loading pipe 223, whereby the sample loading chamber 2 and the substrate 1 (including the amplification reaction chamber 4 within the substrate of the chip) form an independent sealed structure.

FIG. 3B schematically shows that the sample chamber 3 containing the sample to be detected is inserted into the sample loading chamber 2, wherein the sample chamber 3 is in fluid communication with the sample loading chamber 2 through the needle 34, so that the sample solution to be detected in the sample chamber 3 enters the amplification reaction chamber 4 in the substrate through the sample loading chamber 2.

As shown in FIG. 3B, the sample chamber 3 is inserted into the sample loading chamber 2, so that a bottom portion of the lower chamber 32 of the sample chamber 3 is in contact with the upper portion 221 of the sample loading column 22 of the sample loading chamber 2, and the upper sealing rubber plug 35 of the sample chamber 3 is in contact with the lower sealing rubber plug 25 of the sample loading chamber 2. When the sample chamber 3 continues to move downward (i.e. toward the substrate), the sharp tip 341 of the needle can penetrate the upper sealing rubber plug 35, enter and penetrate the lower sealing rubber plug 25, and then be inserted into the sample loading pipe 223 of the sample loading chamber 2, so that the sample chamber 3 is in fluid communication with the sample loading chamber 2. The sample loading chamber 2 and the substrate 1 (including the amplification reaction chamber 4 within the substrate of the chip) initially form an independent sealed structure, wherein the chambers and flow channels therein remain in a vacuum state. When the needle of the sample chamber 3 is inserted into the sample loading pipe 223 of the sample loading chamber 2, the sample chamber 3 is in fluid communication with the sample loading chamber 2. Due to the air pressure, the solution in the sample chamber enters the sample loading chamber 2 and then enters the amplification reaction chamber 4 in the substrate through the liquid flow channel 5 in the substrate. At this time, the spring 23 is compressed.

When the sample chamber 3 is inserted into the sample loading chamber 2 under a thrust and moves downward (toward the substrate), the limiting member 211 is deformed when the sample chamber 3 passes over the limiting member 211, thus not restricting the downward movement of the sample chamber 3.

FIG. 3C schematically shows that the needle of the sample chamber 3 is withdrawn upward to stay in the lower sealing rubber plug 25 after a sufficient amount of the solution for nucleic acid detection has been transferred from the sample chamber 3 to the amplification reaction chamber 4 in the substrate. The sample chamber 3 moves upward pushed by the spring 23. When reaching the limiting member 211, the lower end of the limiting member 211 is clamped into the limiting groove 321, in which case the sample chamber 3 no longer moves upward, so that the tip 341 of the needle stays in the lower sealing rubber plug 25. At this time, the lower sealing rubber plug 25 seals the sample loading pipe 223, and the sample loading chamber 2 and the substrate 1 (including the amplification reaction chamber 4 within the substrate of the chip) form a sealed structure. In the meantime, the lower sealing rubber plug 25 seals the needle 34, so that the sample chamber 3 remains in a sealed state.

In the substrate, the sample preservation solution containing nucleic acids flows to the amplification reaction chamber 4 through the liquid flow channel 5 to redissolve amplification raw materials pre-provided in the amplification reaction chamber, such as freeze-dried particles containing one or more of nucleic acid amplification enzymes, substrates and buffers. Then the nucleic acid amplification reaction is conducted in the amplification reaction chamber to replicate (amplify) typical nucleic acids of the pathogen to be detected and generate corresponding optical signals. Finally, the signals are identified by naked-eye observation, detection through optical device, or the like.

In this embodiment, the substrate is prepared from a rigid material, including but not limited to silica, silicon, quartz, glass, or polymeric materials (such as PDMS, plastic, etc.). The liquid flow channel arranged in the substrate is generally a microfluidic channel with a millimeter-level size. For example, a width of a cross section of the flow channel is approximately 0.1-5 mm.

According to one aspect of the present invention, the substrate and the outer wall of the cylindrical chamber can be prepared from a material that can completely absorb or basically absorb the visible light or fluorescence signal carried by the amplified nucleic acid in order to avoid or minimize the interference from signals in adjacent nucleic acid amplification areas, since the detection of nucleic acid is performed by detecting the target signal (such as fluorescence). Meanwhile, the top/bottom portion of the amplification reaction chamber is prepared from or sealed by a material that does not absorb or basically does not absorb the signal to be detected. Therefore, a signal detection module in the chip or an external detection system can detect the fluorescence signal in each amplification reaction unit through the top/bottom portion of the amplification reaction chamber.

The chip device for detecting nucleic acid in a sample according to the present invention can perform the nucleic acid amplification and signal detection by detecting identifiable markers carried by nucleic acid through any possible method, including but not limited to luminescence in the form of fluorescence or one selected from a group consisting of chemiluminescence, bioluminescence, radioluminescence, electroluminescence, electrochemiluminescence, mechanoluminescence, crystalloluminescence, thermoluminescence, sonoluminescence, phosphorescence and photoluminescence, or enzymatic reactions, radioactivity.

### Embodiment 2 Apparatus for Detecting Nucleic Acid in a Sample

According to one embodiment, the present invention proposes an apparatus for detecting nucleic acid in a sample, which is a POCT apparatus including the chip device as defined and described in Embodiment 1.

The apparatus includes a chip device receiving and motion-controlling system for receiving the chip device and carrying out various treatments of the chip, including heating treatment.

The apparatus may also include a signal detection module for detecting nucleic acid amplification products, such as a fluorescence detection system.

The apparatus further includes a temperature control system for the nucleic acid amplification area of the chip.

The apparatus further includes an analysis and/or output system for nucleic acid amplification results.

The foregoing description is merely illustrative of preferred embodiments of the present invention, and is not intended to restrict the present invention. Any modifications, equivalent substitutions, improvements, and the like falling within the spirit and principles of the present invention are intended to be included within the scope of protection of the present invention.

1.-20. (canceled)

21. A chip device for detecting nucleic acid in a sample, which includes a substrate, a sample loading chamber, a sample chamber detachably connected to the sample loading chamber, and an amplification reaction chamber arranged in the substrate, the sample loading chamber being in communication with the amplification reaction chamber through a liquid flow channel in the substrate,
wherein the sample chamber is configured to receive a sample to be detected, and be inserted into the sample loading chamber to form fluid communication therewith,
wherein the sample to be detected in the sample chamber enters the amplification reaction chamber in the substrate through the sample loading chamber; and
wherein the sample chamber includes a needle, so that the sample chamber is in communication with a sample loading pipe of the sample loading chamber through the needle, allowing a solution in the sample chamber to enter the sample loading chamber.

22. The chip device according to claim 21, wherein a bottom portion of the sample chamber has an upper sealing rubber plug for sealing the needle, and a top portion of the sample loading chamber has a lower sealing rubber plug for sealing the sample loading pipe,
wherein a lower end of the sample chamber is configured to be inserted into the sample loading chamber after the sample chamber and the sample loading chamber are assembled together,
wherein the needle of the sample chamber penetrates the upper sealing rubber plug and the lower sealing rubber plug to be inserted into the sample loading pipe of the sample loading chamber, so that the sample chamber is in fluid communication with the sample loading chamber.

23. The chip device according to claim 22, wherein the sample loading chamber includes a spring which is compressed when the sample chamber is inserted into the sample loading chamber,
wherein a sample loading column fixed to the substrate is arranged in the sample loading chamber, and the sample loading pipe is arranged on a lower portion of the sample loading column, a gap being formed between an outer wall of the sample loading chamber and the sample loading column, for receiving the spring and allowing the lower end of the sample chamber to be inserted.

24. The chip device according to claim 21, wherein the sample loading chamber and the substrate are restored to a sealed state after a sufficient amount of solution for detection from the sample chamber has been transferred to the amplification reaction chamber in the substrate.

25. The chip device according to claim 24, wherein the sample loading chamber and the substrate are restored to the sealed state by withdrawing the needle of the sample chamber from the sample loading pipe of the sample loading chamber,
wherein a tip of the needle of the sample chamber is configured to stay in the lower sealing rubber plug of the sample loading chamber after the needle is withdrawn from the sample loading pipe of the sample loading chamber.

26. The chip device according to claim 25, wherein a limiting member, which is a wedge-shaped member with a protruding lower end, is arranged inside the sample loading chamber, wherein the protruding lower end is deformed when subjected to pressure, and
a limiting groove is arranged at a lower bottom end of the sample chamber, the lower end of the limiting member being clamped into the limiting groove of the sample chamber when the sample chamber moves upward to the limiting member.

27. The chip device according to claim 21, wherein a cap is provided on a top portion of the sample chamber.

28. The chip device according to claim 21, wherein a reagent solution for preserving and extracting the nucleic acid in the sample is added to or pre-provided in the sample chamber, and materials for the nucleic acid amplification reaction are pre-provided in the amplification reaction chamber.

29. The chip device according to claim 21, wherein a pressure sharing chamber, which is arranged downstream of the amplification reaction chamber and within the substrate of the chip, is in communication with the amplification reaction chamber through a pressure sharing gas channel.

30. An apparatus for detecting nucleic acid in a sample, comprising the chip device according to claim 21.

31. The apparatus according to claim 30, wherein the apparatus includes at least one of a chip device receiving system, a signal detection module for detecting nucleic acid amplification products, a temperature control system for a nucleic acid amplification area of the chip device, and an analysis and/or output system for nucleic acid amplification results.

32. The apparatus according to claim 30, wherein the apparatus is a POCT apparatus.

33. A method for detecting nucleic acid in a sample, which is performed by means of the chip device according to claim 21,
wherein the method includes steps of:
(1) adding the sample to the sample chamber of the chip device, so that the nucleic acid in the sample is separated to enter the solution;
(2) inserting the sample chamber into the sample loading chamber to form a fluid communication therebetween, wherein the sample to be detected in the sample chamber enters the amplification reaction chamber in the substrate through the sample loading chamber; and
(3) carrying out the amplification reaction of the nucleic acid in the solution in the amplification reaction chamber.

34. The method according to claim 33, wherein the nucleic acid amplification reaction is an isothermal amplification method.

35. The method according to claim 34, wherein the nucleic acid amplification reaction is loop-mediated isothermal amplification (LAMP) of DNA.

36. The method according to claim 33, wherein the sample chamber includes a needle, through which the sample chamber is in communication with the sample loading pipe of the sample loading chamber, allowing the solution in the sample chamber to enter the sample loading chamber;
wherein an upper sealing rubber plug for sealing the needle is arranged at the bottom portion of the sample chamber, and a lower sealing rubber plug for sealing the sample loading pipe is arranged at the top portion of the sample loading chamber, so that after the sample chamber and the sample loading chamber are assembled together, the lower end of the sample chamber is inserted into the sample loading chamber,
wherein the needle of the sample chamber penetrates the upper sealing rubber plug and the lower sealing rubber plug to be inserted into the sample loading pipe of the sample loading chamber, thereby forming a fluid communication between the sample chamber and the sample loading chamber.

37. The method according to claim 36, wherein after a sufficient amount of solution for detection from the sample chamber has been transferred to the amplification reaction chamber in the substrate, the sample loading chamber and the substrate are restored to a sealed state by withdrawing the needle of the sample chamber from the sample loading pipe of the sample loading chamber.

38. The method according to claim 37, wherein a tip of the needle of the sample chamber is controlled to stay in the lower sealing rubber plug of the sample loading chamber after the needle is withdrawn from the sample loading pipe of the sample loading chamber.

39. The method according to claim 38, wherein a limiting member which is a wedge-shaped member with a protruding lower end, is arranged inside the sample loading chamber,
wherein the protruding lower end is deformed when subjected to pressure, and
a limiting groove is arranged at a lower bottom end of the sample chamber, the lower end of the limiting member being clamped into the limiting groove of the sample chamber when the sample chamber moves upward to the limiting member.

40. The method according to claim 33, wherein identifiable markers carried by the amplified nucleic acid are detected through luminescence in the form of fluorescence or one selected from a group consisting of chemiluminescence, bioluminescence, radioluminescence, electroluminescence, electrochemiluminescence, mechanoluminescence, crystalloluminescence, thermoluminescence, sonoluminescence, phosphorescence and photoluminescence, or enzymatic reactions, radioactivity.
